(19) United States
(12) Patent Application Publication
(10) Pub. No.: US 2010/0286081 A1
(43) Pub. Date: Nov. 11, 2010

(54) 5-AMINO-3-(2',3'-DI-O-ACETYL-BETA-D-RIBOFURANOSYL)-3H-THIAZOLO[4,5-D] PYRIMIDIN-2-OCE SALTS AND CRYSTALLINE FORMS

(75) Inventors: Michael Mutz, Freiberg (DE); Casper Volgel, Binningen (CH)

Correspondence Address: CONNOLLY BOVE LODGE & HUTZ LLP, 1875 EYE STREET, N.W., SUITE 1100, WASHINGTON, DC, 20006, US

(73) Assignee: Anadys Pharmaceuticals Inc.

(21) Appl. No.: 12/301,897

(22) PCT Filed: May 21, 2007
(86) PCT No.: PCT/EP2007/054899

§ 371 Date: Jul. 14, 2010

Related U.S. Application Data
(60) Provisional application No. 60/802,425, filed on May 22, 2006.

(51) Int. Cl. A61K 31/7064; C07H 19/24; A61P 31/14; A61P 31/20

(52) U.S. Cl. 514/43; 536/27.13

(57) ABSTRACT

The invention relates to a maleate salt of 5-amino-3-(2′-3′-di-O-acetyl-beta-D-ribofuranosyl)-3H-thiazolo[4,5-d]pyrimidin-2-one and crystalline forms thereof, their production and usage, and pharmaceutical preparations containing these crystalline forms.

The present invention relates to maleate salt of 5-Amino-3-(2′,3′-di-O-acetyl-beta-D-ribofuranosyl)-3H-thiazolo[4,5-d]pyrimidin-2-one and crystalline forms thereof. Also provided are processes for the preparation thereof, pharmaceutical compositions comprising this salt and crystalline forms thereof and their uses in therapeutic treatment of warm-blooded animals, especially humans.

5-Amino-3-(2′,3′-di-O-acetyl-beta-D-ribofuranosyl)-3H-thiazolo[4,5-d]pyrimidin-2-one can be represented by the following formula

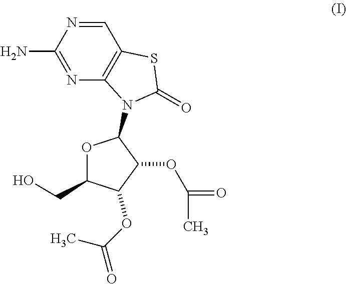

and is known from WO2005/121162, the entire disclosure of which is incorporated by reference, and can be synthesized as described therein.

The free base of 5-Amino-3-(2′,3′-di-O-acetyl-beta-D-ribofuranosyl)-3H-thiazolo[4,5-d]pyrimidin-2-one is an amorphous substance. Prior to the present invention 5-Amino-3-(2′,3′-di-O-acetyl-beta-D-ribofuranosyl)-3H-thiazolo[4,5-d]pyrimidin-2-one had never been recovered in crystalline form. It has now been surprisingly found in accordance with the present invention that under certain conditions crystalline forms can be obtained from the maleate salt of 5-Amino-3-(2′,3′-di-O-acetyl-beta-D-ribofuranosyl)-3H-thiazolo[4,5-d]pyrimidin-2-one. The crystalline forms of the present invention have advantageous properties over the amorphous form of 5-Amino-3-(2′,3′-di-O-acetyl-beta-D-ribofuranosyl)-3H-thiazolo[4,5-d]pyrimidin-2-one e.g. less solvent residue in the ultimate drug substance in whatever form, such as dissolved state, additional purification effect obtained by crystallization, higher stability of the drug substance and easier handling in the production plant.

The free base of 5-Amino-3-(2′,3′-di-O-acetyl-beta-D-ribofuranosyl)-3H-thiazolo[4,5-d]pyrimidin-2-one is a hygroscopic substance. From the chemical structure it is expected that 5-Amino-3-(2′,3′-di-O-acetyl-beta-D-ribofuranosyl)-3H-thiazolo[4,5-d]pyrimidin-2-one is very sensitive to hydrolysis. It has now been surprisingly found in accordance with the present invention that the crystalline forms of the maleate salt are only slightly hygroscopic thus having better storage properties and being easier to process.

The free base of 5-Amino-3-(2′,3′-di-O-acetyl-beta-D-ribofuranosyl)-3H-thiazolo[4,5-d]pyrimidin-2-one has been found to contain some related substances and shows residual solvents and water. Crystalline forms of 5-Amino-3-(2′,3′-di-O-acetyl-beta-D-ribofuranosyl)-3H-thiazolo[4,5-d]pyrimidin-2-one which are essentially pure can be obtained in accordance with the present invention. The term essentially pure in accordance with the present invention is means that the sum of related substances is less than 2% or less than 1%, preferably less than 0.75%, more preferably less than 0.5% and that the residual solvents and water are less than 2% or less than 1%, preferably less than 0.75%, more preferably less than 0.5% and still more preferably less than 0.25% by weight.

In accordance with the present invention it has surprisingly been found that crystalline 5-Amino-3-(2′,3′-di-O-acetyl-beta-D-ribofuranosyl)-3H-thiazolo[4,5-d]pyrimidin-2-one maleate is recovered in at least two polymorphic forms, termed hereinbelow as Form A and Form B. In certain embodiments of the invention, crystalline 5-Amino-3-(2′,3′-di-O-acetyl-beta-D-ribofuranosyl)-3H-thiazolo[4,5-d]pyrimidin-2-one maleate in Form B, which has been found to be a particularly stable polymorphic form, is preferred.

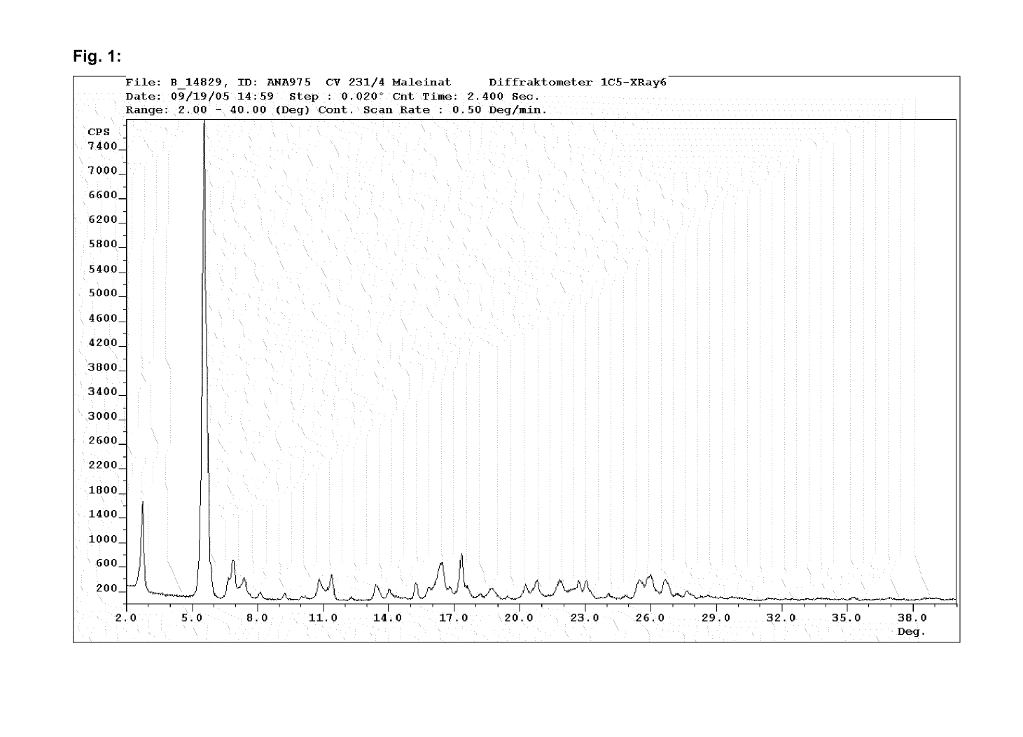

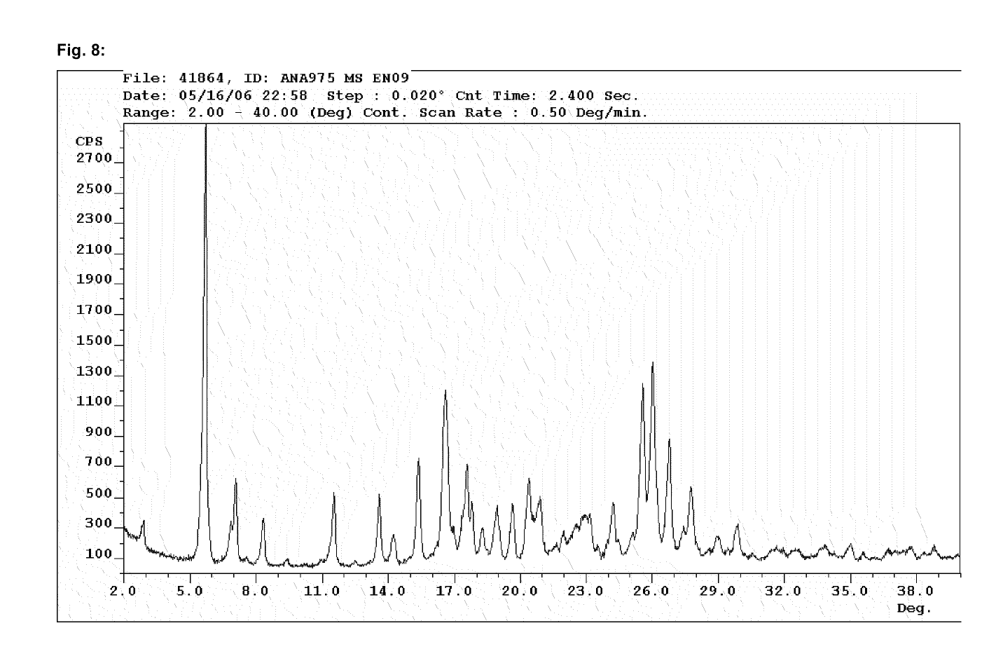

FIG. 1 and FIG. 8 show the X-ray diffraction diagram of a crystalline form of 5-Amino-3-(2′,3′-di-O-acetyl-beta-D-ribofuranosyl)-3H-thiazolo[4,5-d]pyrimidin-2-one maleate hereinafter termed “Form A”. In the X-ray diagram, the angle of diffraction 2theta is plotted on the horizontal axis (x-axis) and the peak intensity on the vertical (y-axis). X-ray powder diffraction patterns are measured on a Scintag X1 diffractometer with Cu Kα radiation source (Kα1 radiation, wavelength λ=1.54060 Angström). A characteristic peak in the X-ray diffraction diagram is observed at an angle of diffraction 2theta of 5.5°, here having a relative intensity of 100%. Further characteristic peaks are observed at 2.7°, 11.4°, 15.3°, 16.4° and 17.3°. More broadly, the A form may be characterized by diffractions peaks at angles of diffraction 2theta of 2.7°, 5.5°, 6.9°, 7.4°, 8.1°, 10.8°, 11.4°, 13.4°, 14.0°, 15.3°, 16.4°, 17.3° or as displayed in FIG. 1 or FIG. 8.

TABLE 1
List of most significant diffraction peaks of
Form A of 5-amino-3-(2′,3′-di-O-acetyl-beta-D-ribofuranosyl)-
3H-thiazolo[4,5-d]pyrimidin-2-one maleate salt

| Position | d-spacing | |
|---|---|---|
| (2-theta) | (Å) | Rel. Intensity |
| 2.73 | 32.235 | medium |
| 5.54 | 15.916 | strong |
| 6.87 | 12.840 | weak |
| 7.38 | 11.966 | weak |
| 8.12 | 10.880 | weak |
| 10.83 | 8.160 | weak |
| 11.38 | 7.768 | weak |
| 13.43 | 6.585 | weak |
| 14.04 | 6.305 | weak |
| 15.25 | 5.805 | weak |
| 16.42 | 5.392 | weak |
| 17.33 | 5.113 | medium |
| 18.64 | 4.756 | weak |
| 20.26 | 4.380 | weak |
| 20.78 | 4.272 | weak |
| 21.83 | 4.068 | weak |
| 25.48 | 3.493 | weak |
| 25.88 | 3.440 | weak |

Accordingly, there is provided a polymorph of the 5-amino-3-(2′,3′-di-O-acetyl-beta-D-ribofuranosyl)-3H-thiazolo[4,5-d]pyrimidin-2-one maleate salt in Form A wherein said crystalline form is characterized by at least one of the following diffraction peaks at angles of diffraction 2theta (±0.5°): 2.7°, 5.5°, 6.9°, 7.4°, 8.1°, 10.8°, 11.4°, 13.4°, 14.0°, 15.3°, 16.4°, 17.3° or at least one characteristic peak shown in FIG. 1 or FIG. 8. As appreciated by the person of skill in the art, the relative intensities of the diffractions can vary depending e.g. on the sample preparation or the instrument used and also, some of the peaks may not always be detectable.

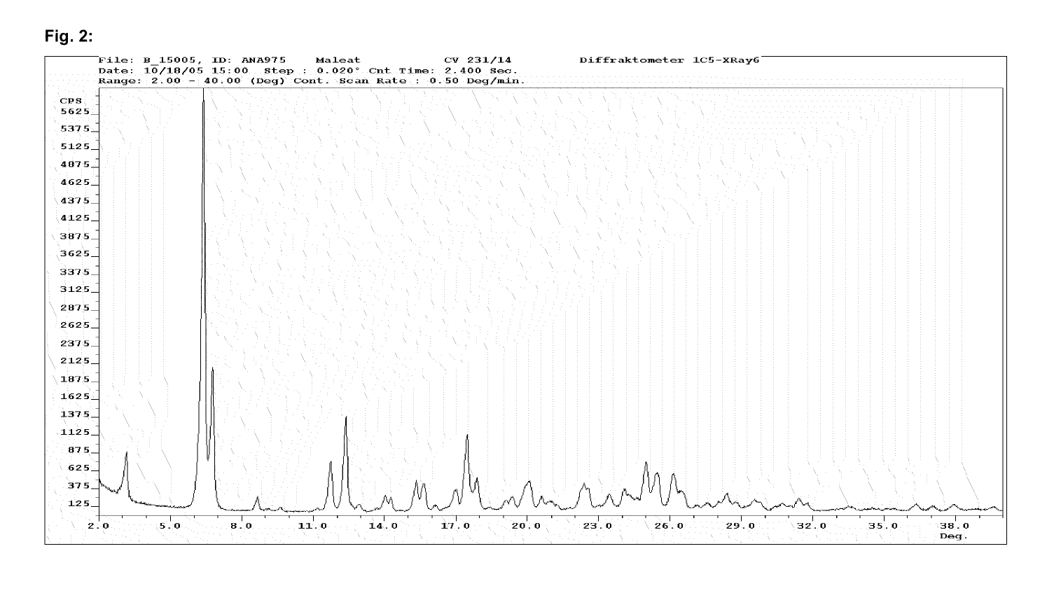

FIG. 2 shows the X-ray diffraction diagram of a crystalline form of 5-Amino-3-(2′,3′-di-O-acetyl-beta-D-ribofuranosyl)-3H-thiazolo[4,5-d]pyrimidin-2-one maleate hereinafter termed “Form B”. The X-ray diagram was recorded as described above. A characteristic peak in the X-ray diffraction diagram is observed at an angle of refraction 2theta of 6.4°, here having a relative intensity of 100%. Further lines are observed at 6.8° and at 12.4° and 17.5°. More broadly, the B form may be characterized by diffractions at angles of diffraction 2theta of 3.2°, 6.4°, 6.8°, 12.4° and 17.5° or as displayed in FIG. 2.

TABLE 2
List of most significant diffraction peaks of Form B
of 5-amino-3-(2′,3′-di-O-acetyl-beta-D-ribofuranosyl)-
3H-thiazolo[4,5-d]pyrimidin-2-one maleate salt

| Position | d-spacing | |
|---|---|---|
| (2-theta) | (A) | Rel. Intensity |
| 3.15 | 27.9841 | weak |
| 6.36 | 13.8748 | strong |
| 6.74 | 13.0915 | medium |
| 12.35 | 7.1592 | medium |
| 17.44 | 5.0801 | medium |

Accordingly, there is provided a polymorph of the 5-amino-3-(2′,3′-di-O-acetyl-beta-D-ribofuranosyl)-3H-thiazolo[4,5-d]pyrimidin-2-one maleate salt in Form B wherein said crystalline form is characterized by at least one of the following diffraction peaks at angles of diffraction 2theta (±0.5°): 3.2°, 6.4°, 6.8°, 12.4° and 17.5° or at least one characteristic peak shown in FIG. 2. As appreciated by the person of skill in the art, the relative intensities of the diffractions can vary depending e.g. on the sample preparation or the instrument used and also, some of the above peaks may not always be detectable.

In accordance with the present invention, the observed angle of diffraction 2theta can deviate ±0.1°, ±0.2°, ±0.3°, ±0.5°, preferably up to ±10% of the above angles of refraction.

Form A can also be characterized by melting onset temperature of about 95° C. to 115° C. or about 100° C. to 110° C., e.g. about 105° C., Form B can be characterized by a melting peak in the range of about 110° C. to 140° C. or about 120° C. to about 140° C., e.g. with a melting onset temperature of about 126° C. or about 131° C. Melting points can be determined by means of a DSC thermogram using a Mettler-Toledo DSC822. DSC (“differential scanning calorimetry”) is the technique of dynamic differential calorimetry. Using this technique, the melting temperature of the Form A and B can be measured by heating the samples until a thermal, i.e. an endothermic reaction is detected by means of ultrasensitive sensors. The melting points indicated in this text are determined using a Mettler-Toledo DSC822 apparatus, about 1 to 3 mg of each sample being measured in an aluminium crucible with a perforated lid under an atmosphere of nitrogen at a heating rate of 10° C./min (starting at 30° C.). As appreciated by the skilled person melting temperatures may differ depending e.g. on the purity of the sample measured. Deviations of for instance ±10° C. may not be uncommon.

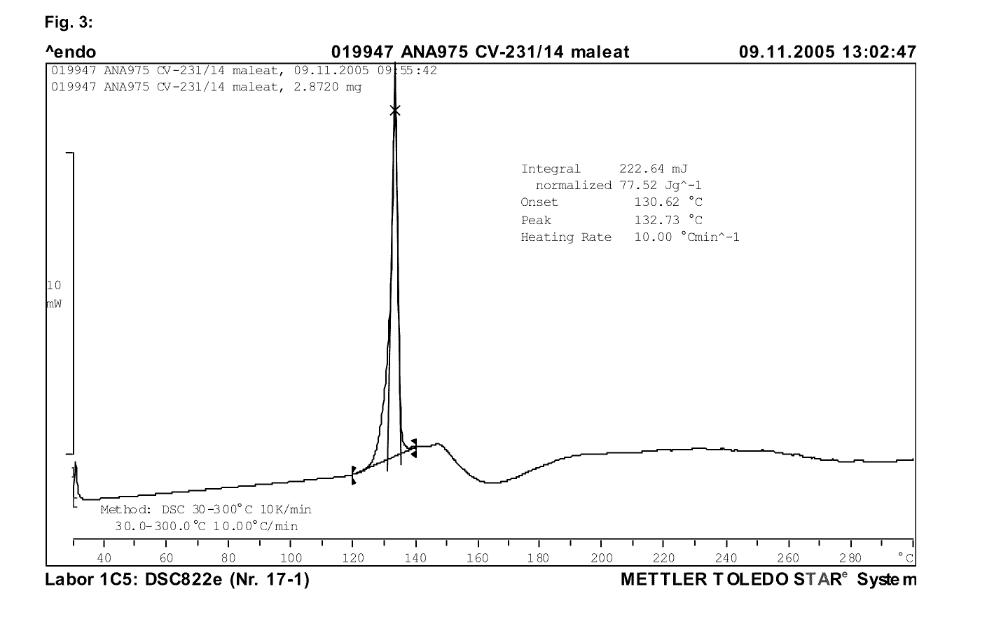

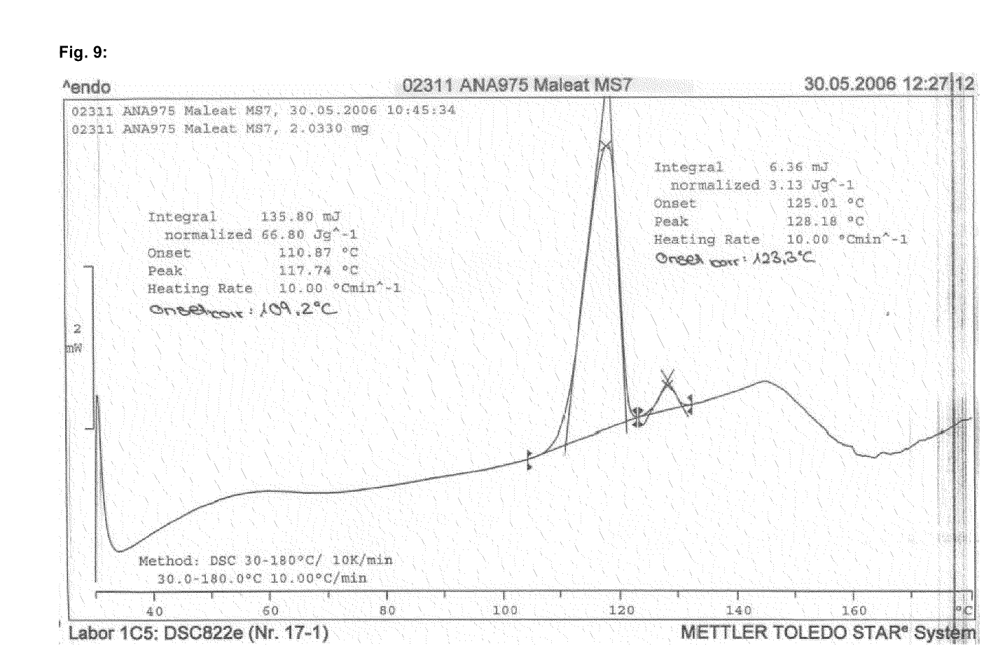

FIG. 3 shows the DSC curve of Form B of the crystalline 5-Amino-3-(2′,3′-di-O-acetyl-beta-D-ribofuranosyl)-3H-thiazolo[4,5-d]pyrimidin-2-one maleate. FIG. 9 shows the DSC curve of Form A.

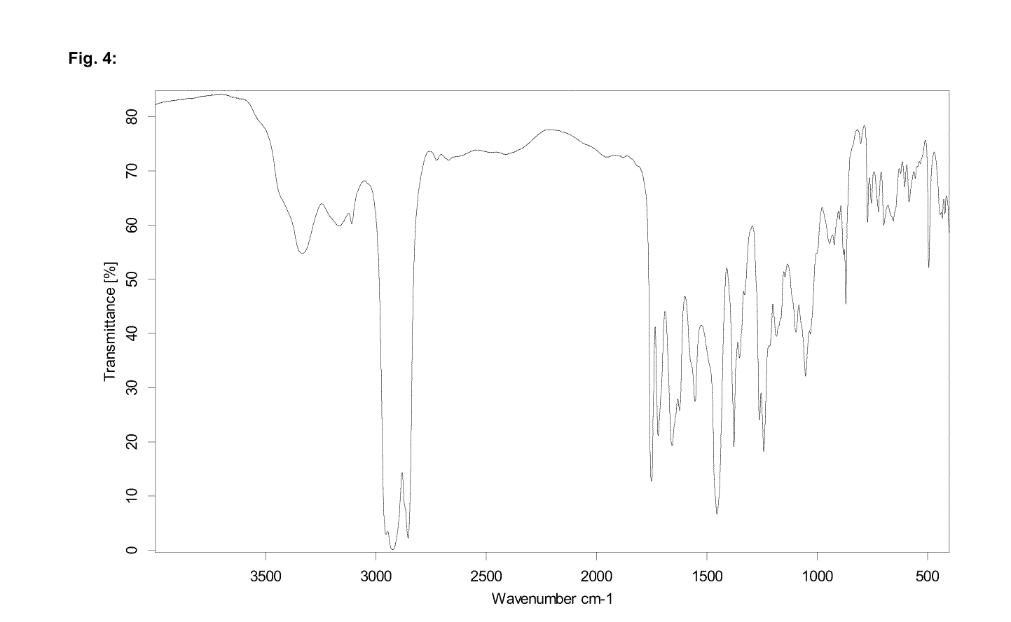

FIG. 4 shows the FT-IR spectrum of Form B. The FT-IR spectrum was recorded using a Burker IFS-55. The sample was prepared in nujol an placed between two KBr plates. The Form B is characterized by the following major IR bands 2925, 2854, 1750, 1454. More broadly by the following IR bands: ˜3331, 3166, 3109, 2925, 2854, ˜2500, 2000 (broad), 1750, 1721, 1658, 1624, 1553, 1454, 1378, 1097, 1053, 803, 772, 755.

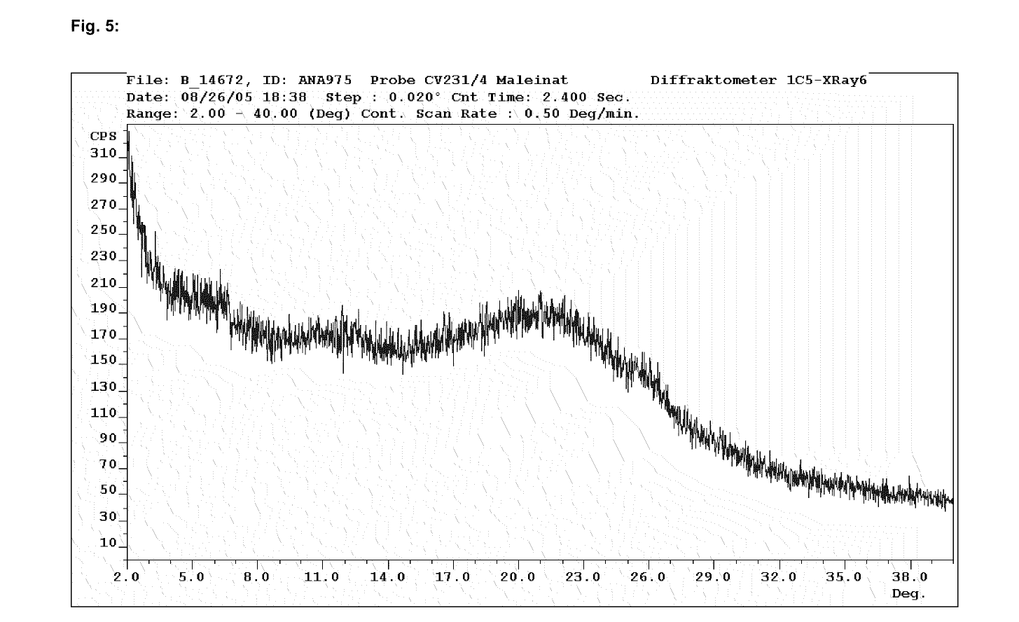

FIG. 5 shows the X-ray powder diffraction pattern of amorphous form of maleate salt.

In one embodiment, the crystalline form B of 5-Amino-3-(2′,3′-di-O-acetyl-beta-D-ribofuranosyl)-3H-thiazolo[4,5-d]pyrimidin-2-one maleate are not hydrated, i.e. the anhydrate.

In accordance with one aspect, the present invention provides a process for the preparation of a maleate salts of the invention which comprises reacting the compound of formula I in free base form with an appropriate maleic acid form and recovering from the reaction mixture the resultant salt. The process of the invention may be effected in conventional manner, e.g. by reaction in an appropriate inert solvent such as TBME, methanol, ethanol or isopropanol.

In accordance with another aspect of the invention, a process for the crystallization of 5-Amino-3-(2′,3′-di-O-acetyl-beta-D-ribofuranosyl)-3H-thiazolo[4,5-d]pyrimidin-2-one maleate is provided. The precise conditions under which crystals are formed may now be empirically determined and a number of methods are suitable in practice, including the crystallization conditions as described in Examples 1, 2 and 4.

Crystallization-inducing conditions normally involve the use of an appropriate crystallization-inducing solvent, such as t-butylmethylether (TBME), methanol, ethanol, isopropanol or water or mixtures thereof. Conveniently, the amorphous compound is dissolved in the solvent at a temperature of normally at least 10° C. The solution may be produced by dissolving in a solvent any one or more of amorphous forms of the compound, and solvates thereof, such as hydrates, methanolates, ethanolates, or isopropanolates. Crystals may then be formed by conversion from solution, crystallization taking place at a temperature of between about 0° C. and the boiling point of the solvent. The dissolution and crystallization may be carried out in various conventional ways. For instance, amorphous compound may be dissolved in a solvent or a mixture of solvents in which it is readily soluble at elevated temperatures but in which it is only sparingly soluble at lower temperatures. Dissolution at elevated temperature is followed by cooling during which the desired crystals crystallize out of solution. A cooling and reheating step may be carried out several times, e.g. at least once, at least twice, at least 3×, at least 5×. The cooling and reheating temperatures are e.g. at least 5° C., at least 10° C. or at least 15° C. The low temperature of the cooling/heating cycles may e.g. be less than 15° C., less than 10° C., less than 5° C. or less than 0° C., whereas the high temperature may e.g. be at least 15° C., at least 20° C., at least 25° C. or at least 30° C.

Mixed solvents comprising a good solvent in which the compound is readily soluble, preferably, in amounts of at least 1% by weight at 30° C., and a poor solvent in which it is more sparingly soluble, preferably in amounts of not more than about 0.01% by weight at 30° C., may also be employed provided that crystallization from the mixture at a reduced temperature, of normally at least about, 0° C., is possible using the selected solvent mixture.

Alternatively, the difference in solubility of the crystals in different solvents may be used. For example, the amorphous compound may be dissolved in a good solvent in which it is highly soluble such as one in which it is soluble in amounts of at least 1% by weight at about 30° C. and the solution subsequently mixed with a poor solvent in which it is more sparingly soluble, such as one in which it is soluble in amounts of not more than about 0.01% by weight at about 30° C. Thus, the solution of the compound in the good solvent may be added to the poor solvent, while maintaining normally a temperature in excess of about 0° C., or the poor solvent may be added to the solution of the compound in the good solvent, again while normally maintaining a temperature in excess of about 0° C. Examples of good solvents may include lower alcohols, such as methanol, ethanol and isopropanol, or acetone. An example of a poor solvent is e.g. water. Preferably, crystallization is effected at a temperature in the range of about 0° C. to about 40° C.

In an alternative embodiment of the process of the invention, solid amorphous compound is suspended at a temperature of normally at least about 0° C. in a solvent in which it is incompletely soluble, preferably only sparingly soluble, at that temperature. A suspension results in which particles of solid are dispersed, and remain incompletely dissolved in the solvent. Preferably the solids are maintained in a state of suspension by agitation e.g. by shaking or stirring. The suspension is kept at a temperature of normally about 0° C. or higher in order to effect a transformation of the starting solids into crystals. The amorphous solid compound suspended in a suitable solvent may be a solvate, e.g. hydrate, methanolate or ethanolate. The amorphous powder may be derived by drying a solvate.

It is possible to add “seeds” of crystalline material (if available) to the solution in order to induce crystallization.

In one aspect the present invention provides pharmaceutical composition comprising an effective amount of a crystalline form of 5-Amino-3-(2′,3′-di-O-acetyl-beta-D-ribofuranosyl)-3H-thiazolo[4,5-d]pyrimidin-2-one maleate and a suitable carrier.

One embodiment provides methods of preventing or treating infections of a warm-blooded animal, especially a human, by a pathogenic organism comprising administering an effective amount of amorphous, or a crystalline form of, 5-Amino-3-(2′,3′-di-O-acetyl-beta-D-ribofuranosyl)-3H-thiazolo[4,5-d]pyrimidin-2-one maleate. In a preferred embodiment the pathogenic organism is a bacterial, fungal or viral infection disclosed in WO2005/121162, in another preferred embodiment a viral infection caused by adenovirus, cytomegalovirus, hepatitis A virus (HAV), hepatitis B virus (HBV), flaviviruses including Yellow Fever virus and hepatitis C virus (HCV), herpes simplex type 1 and 2, herpes zoster, human herpesvirus 6, human immunodeficiency virus (HIV), human papilloma virus (HPV), influenza A virus, influenza B virus, measles, parainfluenza virus, poliovirus, poxvirus (including smallpox and monkeypod virus), rhinovirus, respiratory syncytial virus (RSV), multiple families of viruses that cause hemorrhagic fevers, including the Arenaviruses (LCM, Junin virus, Machup virus, Guanarito virus, and Lassa Fever), the Bunyaviruses (Hanta viruses and Rift Valley Fever) and Filoviruses (Ebola and Marburg virus), a range of viral encephalitides including West Nile virus, LaCrosse virus, California Encephalitis virus, Venezuelan Equine Encephalitis virus, Eastern Equine Encephalitis virus, Western Equine Encephalitis virus, Japanese Encephalitis virus, Kysanur Forest virus, and tickborne viruses such as Crimean-Congo Hemorrhagic fever virus. Particularly preferred are HBV and HCV. Another embodiment provides methods of modulating immune cytokine activities of a warm-blooded animal, especially a human, comprising administering an effective amount of a crystalline form of 5-Amino-3-(2′,3′-di-O-acetyl-beta-D-ribofuranosyl)-3H-thiazolo[4,5-d]pyrimidin-2-one maleate. Also provided is a crystalline form of 5-Amino-3-(2′,3′-di-O-acetyl-beta-D-ribofuranosyl)-3H-thiazolo[4,5-d]pyrimidin-2-one maleate for use in medicine. Also provided is the use of a crystalline form of 5-Amino-3-(2′,3′-di-O-acetyl-beta-D-ribofuranosyl)-3H-thiazolo[4,5-d]pyrimidin-2-one maleate for the manufacture of a medicament for the treatment of an infection by a pathogen, especially a virus, e.g. HCV or HBV. Further provided is the use of a crystalline form of 5-Amino-3-(2′,3′-di-O-acetyl-beta-D-ribofuranosyl)-3H-thiazolo[4,5-d]pyrimidin-2-one maleate for the manufacture of a medicament modulating immune cytokine activities of a warm-blooded animal.

The present invention further includes:
a pharmaceutical composition comprising a salt or a crystalline salt of the invention together with at least one pharmaceutically acceptable carrier or diluent;
a pharmaceutical composition comprising the compound of formula I in free form or pharmaceutically acceptable salt form other than a maleic acid addition salt form, whenever prepared from a salt or a crystalline salt of the invention;
a salt or a crystalline salt of the invention for use as a pharmaceutical;
a salt or a crystalline salt of the invention for use in the preparation of a medicament;
a salt or a crystalline salt of the invention whenever prepared by a process as defined above;
a salt or a crystalline salt of formula I in free base form or salt form other than a maleic acid addition salt form, whenever prepared from a salt or a crystalline salt of the invention;
the use of a compound of the invention in the preparation of a medicament for the treatment, e.g. orally or intravenously, of diseases susceptible of therapy with the salt or the crystalline salt of formula I in free base form or salt form, such as viral diseases;
a process for the preparation of a pharmaceutical composition which comprises mixing a salt or a crystalline salt of the invention together with at least one pharmaceutically acceptable carrier or diluent; and
a method for the prophylactic or curative treatment of viral diseases such as HCV or HBV infection, comprising administration of a therapeutically effective amount of a salt or a crystalline salt of the invention to a subject in need of such treatment.

The crystalline forms of the present invention are synthesized in accordance with the following examples which are illustrative without limiting the scope of the present invention.

### EXAMPLE 1

### Crystalline 5-Amino-3-(2′,3′-di-O-acetyl-beta-D-ribofuranosyl)-3H-thiazolo[4,5-d]pyrimidin-2-one maleate (Form A)

404 mg 5-amino-3-(2′,3′-di-O-acetyl-beta-D-ribofuranosyl)-3H-thiazolo[4,5-d]pyrimidin-2-one base are dissolved in 12 ml TBME at RT then 122 mg Maleic acid dissolved in 1 ml Ethanol are added. This gives a clear solution. By cooling with external temp.=−18° C. amorphous 5-amino-3-(2′,3′-di-O-acetyl-beta-D-ribofuranosyl)-3H-thiazolo[4,5-d]pyrimidin-2-one maleate precipitates out and is removed by filtration. The precipitate is washed with 5 ml TBME, and the washing added to the mother liquor. By standing for several weeks in a fridge at 5° C. a small amount of 5-amino-3-(2′,3′-di-O-acetyl-beta-D-ribofuranosyl)-3H-thiazolo[4,5-d]pyrimidin-2-one maleate form A crystallize out of the combined mother and wash liquor.

### EXAMPLE 2

### Crystalline 5-Amino-3-(2′,3′-di-O-acetyl-beta-D-ribofuranosyl)-3H-thiazolo[4,5-d]pyrimidin-2-one maleate (Form B)

In a 10 l double walled vessel, 303 g 5-amino-3-(2′,3′-di-O-acetyl-beta-D-ribofuranosyl)-3H-thiazolo[4,5-d]pyrimidin-2-one base are dissolved in 10 l of TBME at IT=25° C. Then a solution of 97 g Maleic acid in 820 ml Ethanol are added within 30 min at IT=25° C. At the beginning of this addition a precipitation is formed, which dissolves towards the end of the Addition. Then IT is lowered to 20° C. and the clear solution is seeded with 30 mg 5-amino-3-(2′,3′-di-O-acetyl-beta-D-ribofuranosyl)-3H-thiazolo[4,5-d]pyrimidin-2-one maleat salt suspended in 15 ml TBME (2 min ultrasound treatment). The resulting suspension is aged by the following t-program: 3* cooling to 5° C. and reheating to 20° C. with 1° C./h. After the last cooling step the suspension is filtered and washed with 1 l of TBME. Drying over night in a vacuum oven at 40° C. delivers 307.42 g 5-amino-3-(2′,3′-di-O-acetyl-beta-D-ribofuranosyl)-3H-thiazolo[4,5-d]pyrimidin-2-one maleate (78% of theory) as a white crystalline solid, which according to xrpd contains exclusively form B.

### EXAMPLE 3

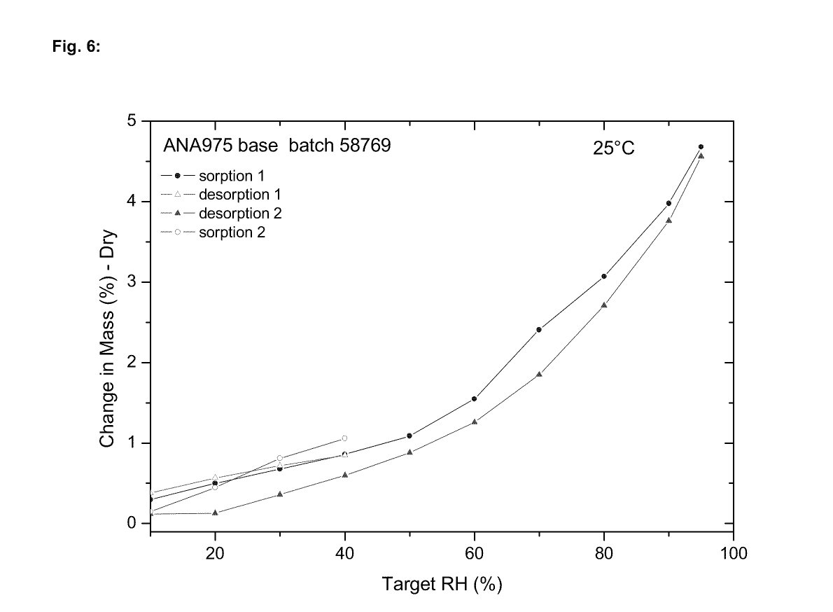

### Water Sorption Curve of 5-amino-3-(2′,3′-di-O-acetyl-beta-D-ribofuranosyl)-3H-thiazolo[4,5-d]pyrimidin-2-one Amorphous Base (FIG. 6)

| DVS analysis at 25° C. | Weight change in % |
|---|---|
| 0-20% r.h.: | 0.50 |
| 0-40% r.h.: | 0.86 |
| 0-60% r.h.: | 1.55 |
| 0-80% r.h.: | 3.07 |
| 0-95% r.h.: | 4.68 |

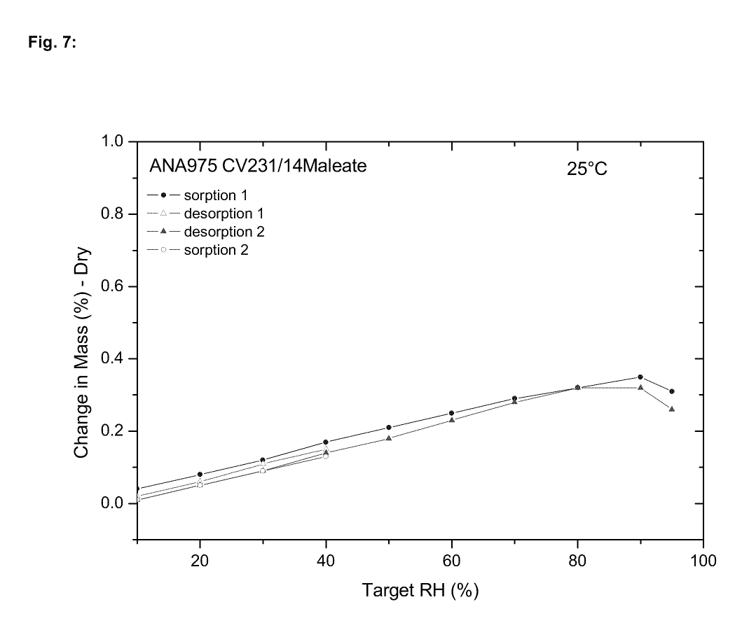

Water Sorption Curve of 5-amino-3-(2′,3′-di-O-acetyl-beta-D-ribofuranosyl)-3H-thiazolo[4,5-d]pyrimidin-2-one Maleate Salt (FIG. 7)

| DVS analysis at 25° C. | Weight change in % |
|---|---|
| 0-20% r.h.: | 0.08% |
| 0-40% r.h.: | 0.17% |
| 0-60% r.h.: | 0.23% |
| 0-80% r.h.: | 0.32% |
| 0-95% r.h.: | 0.31% |

### EXAMPLE 4

### Crystalline 5-Amino-3-(2′,3′-di-O-acetyl-beta-D-ribofuranosyl)-3H-thiazolo[4,5-d]pyrimidin-2-one maleate (Form A)

1.27 g of 5-amino-3-(2′,3′-di-O-acetyl-beta-D-ribofuranosyl)-3H-thiazolo[4,5-d]pyrimidin-2-one base are dissolved in 12 ml of TBME and 2 ml of Ethanol at IT=25° C. to yield a clear solution. Then a solution of 1.21 g Maleic acid in 3 ml Ethanol and 5 ml of TBME is prepared at IT=25° C. The two solutions are mixed at IT=25° C. to yield a cloudy solution which becomes clear on stirring. The clear solution is transferred to 4-6° C. and kept at this temperature for 10-12 hours. The suspension is filtered and the solid dried over night in a vacuum oven at 40° C./2-10 mbar to yield 1.59 g of 5-amino-3-(2′,3′-di-O-acetyl-beta-D-ribofuranosyl)-3H-thiazolo[4,5-d]pyrimidin-2-one maleate (96% of theory) as a white crystalline solid, which according to xrpd contains form A.

1. A maleate salt of 5-Amino-3-(2′,3′-di-O-acetyl-beta-D-ribofuranosyl)-3H-thiazolo[4,5-d]pyrimidin-2-one.

2. A salt according to claim 1 in amorphous form.

3. A maleate salt of 5-Amino-3-(2′,3′-di-O-acetyl-beta-D-ribofuranosyl)-3H-thiazolo[4,5-d]pyrimidin-2-one in crystalline form.

4. A salt according to claim 3 in crystalline form A.

5. A salt according to claim 3 in crystalline form B.

6. A salt according to claim 3 in crystalline form wherein the crystalline form is a mixture of Form A and B.

7. A crystalline salt according to claim 3, which shows on X-ray diffraction a peak at an angle of refraction 2theta of 5.5°±0.5°.

8. A crystalline salt according to claim 3, which shows on X-ray diffraction at least one peak at an angle of refraction 2theta of 2.7°, 5.5°, 6.9°, 7.4°, 8.1°, 10.8°, 11.4°, 13.4°, 14.0°, 15.3°, 16.4° or 17.3° (±0.5°); or at least one peak displayed in FIG. 1 or FIG. 8 (±0.5°).

9. A crystalline salt according to claim 3, which shows on X-ray diffraction a peak at an angle of refraction 2theta of 6.4°±0.5°.

10. A crystalline salt according to claim 3, which shows on X-ray diffraction at least one peak at an angle of refraction 2theta of 3.2°, 6.4°, 6.8°, 12.4° or 17.5° (±0.5°); or as displayed in FIG. 2 (±0.5°).

11. A salt or crystalline salt according to claim 1 which is present in essentially pure form.

12. A process for the preparation of a maleate salt of 5-amino-3-(2′,3′-di-O-acetyl-beta-D-ribo furanosyl)-3H-thiazolo[4,5-d]pyrimidin-2-one according to claim 1 comprising reacting 5-amino-3-(2′,3′-di-O-acetyl-beta-D-ribo furanosyl)-3H-thiazolo[4,5-d]pyrimidin-2-one with malic acid and recovering from the reaction mixture the resultant salt.

13. A process for the preparation of a crystalline maleate salt of 5-amino-3-(2′,3′-di-O-acetyl-beta-D-ribofuranosyl)-3H-thiazolo[4,5-d]pyrimidin-2-one according to claim 2 comprising appropriately converting amorphous maleate salt of 5-Amino-3-(2′,3′-di-O-acetyl-beta-D-ribofuranosyl)-3H-thiazolo[4,5-d]pyrimidin-2-one from a solution thereof under crystallization-inducing conditions.

14. A process for the preparation of a crystalline maleate salt of 5-amino-3-(2′,3′-di-O-acetyl-beta-D-ribofuranosyl)-3H-thiazolo[4,5-d]pyrimidin-2-one according to claim 2 comprising the steps of dissolving 5-amino-3-(2′,3′-di-O-acetyl-beta-D-ribofuranosyl)-3H-thiazolo[4,5-d]pyrimidin-2-one and maleic acid in an appropriate solvent, optionally seeding the solution with maleate salt of 5-amino-3-(2′,3′-di-O-acetyl-beta-D-ribofuranosyl)-3H-thiazolo[4,5-d]pyrimidin-2-one, and at least one cooling of the solution for at least 5° C. and at least one reheating of the solution for at least 5° C.

15. A process for the preparation of a crystalline maleate salt of 5-amino-3-(2′,3′-di-O-acetyl-beta-D-ribofuranosyl)-3H-thiazolo[4,5-d]pyrimidin-2-one according to claim 3 comprising the step of crystallization or re-crystallization a maleate salt of 5-amino-3-(2′,3′-di-O-acetyl-beta-D-ribo furanosyl)-3H-thiazolo[4,5-d]pyrimidin-2-one in a solution comprising TBME.

16. A pharmaceutical composition comprising a maleate salt or a crystalline form of a maleate salt according to claim 1.

17. A method of treating an infection by a pathogen, comprising administering to a patient in need a therapeutically effective amount of a maleate salt or a crystalline form of a maleate salt according to claim 1.

18. A maleate salt or a crystalline form of a maleate salt according to claim 1 for use in medicine.

19. Use of a maleate salt or crystalline form of a maleate salt according to claim 1 for the manufacture of a medicament for the treatment of an infection by a pathogen.

20. The use of claim 19 wherein the pathogen is a virus an in particular HCV or HBV.
